(19) United States
(12) Patent Application Publication
(10) Pub. No.: US 2002/0088467 A1
(43) Pub. Date: Jul. 11, 2002

(54) Mind personality transfer method

(76) Inventor: Izumi Arai, Tokyo (JP)

Correspondence Address: JACOBSON HOLMAN PLLC, 400 SEVENTH STREET N.W., SUITE 600, WASHINGTON, DC, 20004, US

(21) Appl. No.: 09/985,698

(22) Filed: Feb. 25, 2002

Related U.S. Application Data
(63) Continuation of application No. PCT/JP01/01586, filed on Mar. 3, 2001.

(30) Foreign Application Priority Data
Mar. 2, 2000 (JP) 115905

(51) Int. Cl. A61B 19/00

(52) U.S. Cl. 128/898

(57) ABSTRACT

This invention can permanently in the future develop minds and personalities of creatures which were considered in the past to decay and become extinct owing to ageing and death, by transferring minds and personalities of creatures to new clone bodies, with preserving self-identity. This invention is particularly valid in the case that an invalid has little chance of recovery due to serious multiple organ failures, in the case that general prostration or severe senility of the whole body makes urgent organ exchanges ineffective, in the case that it will not be long before an individual moves to new clone bodies on some other grounds, etc. My invention is the repetitive processes of transferring central nervous systems and other systems of individuals and old clone bodies to new clone bodies.

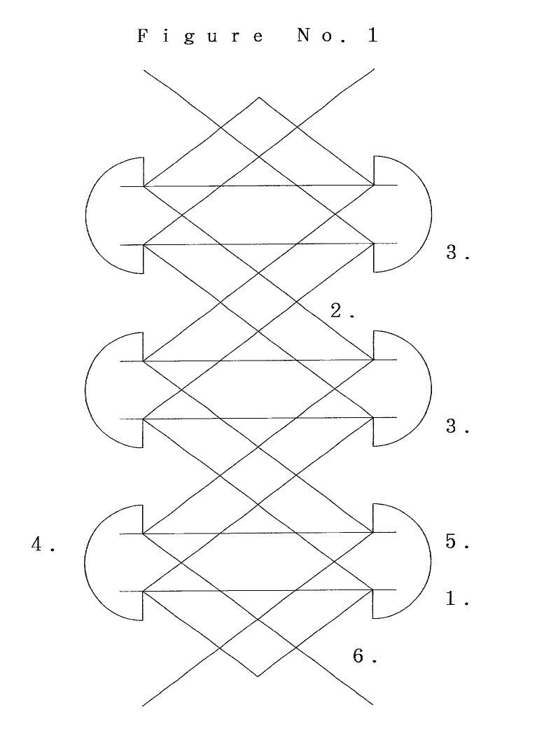

Besides the process of transferring central nervous systems themselves to new clone bodies, as FIG. 1 shows, by inputting memories retained in original individuals and clone bodies to central nervous systems of new clone bodies beforehand, creatures can obtain existence bases of themselves in succession, because memory itself can be regarded as the independent personality.

Combining freely a great many of organs such as central nervous systems, peripheral nervous systems, and other systems, can enlarge memory capacities. Moreover, by establishing new active pathways, the originality worth of oneself can be formed. Remaking gray matters into chips such as semiconductors, various integrated circuits, etc., makes nervous systems light and convenient. The information interchange between lots of brains beyond a tiny skull and the construction of new nervous systems can evolve central nervous systems as a whole.

Creatures can maintain self-identity even if constituent elements of succeeding creatures are different from those of preceding creatures, because ceatures always exchange their constituent elements by respiration and metabolism.

Furthermore, the consciousness of oneself can maintain continuity in the case that the infant self is lasting to the adult self, in the case that a particular self can put on weight and can lose weight, and in the case that the organs of oneself were injured in diseases, accidents, etc., and the self gets one's health back again. Namely, ceatures can transfer themselves in succession to new clone bodies continuously and spaciously, even if succeeding creatures have different shapes, sizes, functions, constituent elements, etc.

Therefore, with keeping self-continuity, I can create the existence of multi-arms and multi-legs, by making the most of lots of other systems in addition to nervous systems simultaneously. And, by obtaining various new organs, I can extend individual faculties of creatures diversely and remarkably.

This is a continuation of PCT/JP01/01586 filed Mar. 1, 2001 and published in Japanese.

### THE FIELD OF MY INVENTION

This invention belongs to the field of biology and life science.

### PAST WAYS OF THINKING

So far, minds and personalities of creatures were thought to collapse and die out, owing to ageing and serious lesions of the body.

### THE POINT OF MY INVENTION

This invention transfers minds and personalities of individuals and clone bodies of creatures to new clone bodies. And thus I can make creatures continue development permanently, with ensuring creatures existence bases of themselves. The sift of minds and personalities of creatures to fresh clone bodies can maintain formal identity and substantial development of the individual forever.

My invention is especially effective in the case that an invalid has little chance of recovery due to serious multiple organ failures, in the case that general prostration or severe senility of the whole body makes urgent organ exchanges ineffective, and in the case that it will not be long before an individual shifts to new clone bodies on some other grounds.

### CONCISE EXPLANATIONS OF MY INVENTIVE FIGURES

The first FIGURE is a picture overlooking central nervous systems which combine in order to transfer minds and personalities and to amplify information. Brains, whose gene types are same, interchange various pieces of information one another across the boundaries of a tiny skull. Therefore, brains produce the further excellent brains which are gifted with newer engrams. In this way these whole central nervous systems are making remarkable progress. 1. individual brains. 2. commissural fibers. 3. clone brains. 4. the right. 5. the left. 6. the front.

### THE EFFECTIVE METHOD OF MY INVENTION

I will explain this invention in detail.

In order to transfer minds and personalities in individuals and clone bodies of creatures to new clone bodies, and to maintain formal identity and substantial development of the individual eternally, this invention makes it possible to obtain immortal lives by repeating the processes of transferring some parts of an individual, especially parts with central nervous systems which take leading parts, to new clone bodies.

I shift central nervous systems in individuals and clone bodies of creatures to new clone bodies. Thereby, an individual can get more excellent and newer bodies. And so an individual can keep up and develop spirit and character of oneself.

In the case of no injury in the head, it is important to secure blood flows from arch of aorta to internal and external iliac arteries. Then, it is suitable to pump 15% of total cardiac output at a velosity of about 700 milliliters per minute, through brachiocephalic trunk into right common carotid and vertebral arteries, and into left common carotid and vertebral arteries. Systems of external carotid arteries and some related arteries are useful for making blood reservoirs. It is desirable to deal with variations and anomalies of blood vessels each time. Cerebral perfusion pressure is from 60 to 160 mmHg. The partial pressure of oxygen in the arterial blood should be over 50 mmHg. Plasma glucose concentration is from 70 to 120 milligrams per deciliter. The body temperature may be lowered to 16 degrees centigrade. It is important to preserve radicular arteries, including the artery of Adamkiewicz, etc. It is essential to prevent ischemia. Blood flow must be regained in less than 20 seconds after ischemia should occur by any chance. The faces, including eyes, noses, etc., have another blood flows from systems of internal carotid arteries as well as from systems of external carotid arteries. Therefore, these parts, which adhere to cranial bones, are suitable for forming and repairing plastically as the need arises, after the transference to new clone bodies.

By securing blood flows from subclavian, jugular, and brachiocephalic veins to internal and external iliac veins on both sides, and by securing lymph flows from lumbar lymph nodes and intercostal lymph nodes to thoracic duct and right lymphatic duct, body fluids return smoothly to the pump, which keep good circulation.

Spinal cords are suitable for transferring to clone bodies together with spinal ganglia. It is appropriate to move with vertebrae. However, when something like severe osteoporosis is found, it is desirable to separate the parts of the outer layer of spinal dura mater to epineurium wrapping spinal ganglia from vertebral bodies and vertebral arches, with maintaining smooth circulation so as not to cause edema. It is also pertinent to move and detach the remainder from central nervous systems so that brains and spinal cords are not injured.

In the case of some damage in the head, covering up the deficits with materials including artificial ones, is suitable in shifting central nervous systems. When marked damage is found in cranial bones, cranial nerves, etc., at least contents in dura mater can be transferred. Reducing edema and repairing injuries with clone nerve cells, clone neuroglia, clone tissues, cerebrospinal fluids, etc., are needed when brains and spinal cords have injuries in themselves. In the case that these injuries are grave, it is necessary to repair them with clone nuclei and clone gray matter and to make variable synapses learn and memorize lost functions.

Hippocampal formation retains new pieces of information temporarily and reproduces these information. The whole brain is necessary for fixation of remote memories and long-term retention. Declarative memories need limbic system including hippocampal formation, entorhinal cortex, formix, etc. Procedural memories need cerebellum and brainstem including olivary nuclei, etc. Commissural fibers, such as corpus callosum, anterior part of commissura anterior, posterior part of commissura anterior, commissura hippocampi, commissura posterior, commissura habenularum, commissurae supraopticae (commissura Guddeni, commissura Meynerti, commissura Ganseri), etc., can combine individual brains with clone brains since an individual is still alive.

Commissural fibers combining neuron groups of the same gene types transmit information from the right to the left, and from the left to the right. It is nice to use together new brains into which information has extended and healthy original brains of the individual. New brains should succeed to old brains, in the case that old brains are severely injured by senility, brain atrophy, etc., and have no hope of recovery even by supplementation on the level of cells and tissues. New brains may settle down to the primary body whose regeneration is possible. And new brains may also lead a comfortable life in new clone bodies. When new brains return to the primary body, it is convenient to supplement as many parts of new clone bodies as possible to the primary body. When new brains live in new clone bodies, the necessary minimun of the primary body can be moved to new clone bodies if a new brain want to use the original parts of the old body. For a certain period of time, putting both old and new brains into operation simultaneously and transferring functions gradually from old brains to new brains make it possible to realize self-continuity as time passes, without anxiety.

Using together a lot of brains by combining a great many of specific areas and hemispheres with commissural fibers, association fibers, etc., can increase memory capacity, with being free from small volume of the skull, and can even establish new active pathways. Remaking gray matters into chips such as semiconductors, various integrated circuits, etc., makes nervous systems light and convenient. By securing plenty of nervous systems, such as basal ganglia, various nuclei, cerebella, brainstems, ganglia, etc., and by making the most of lots of other systems in addition to nervous systems at the same time, I can create the existence of multi-arms and multi-legs, which enlarges spacial self-continuity. Fresh brains can be brought up, while coming into subsidiary operation, with carrying these immature brains on one's back. Free combinations of brains brought up in various environments broaden the world of one's own, which produce latest brains that have newly composed engrams. Incorporating these latest brains into central nervous systems once more leads to infinite development.

### THE SIGNIFICANCE IN MY INVENTION

In this way, my invention permanently in the future develops minds and personalities of creatures which were considered in the past to decay and become extinct owing to senility and death, by transferring minds and personalities of creatures to new clone bodies, with maintaining self-identity. This invention is particularly valid in the case that an invalid has little chance of recovery due to serious multiple organ failures, in the case that general prostration or severe senility of the whole body makes urgent organ exchanges ineffectual, in the case that it will not be long before an individual moves to new clone bodies on some other grounds, etc. Transferring central nervous systems and other systems in individuals and clone bodies of creatures to new clone bodies can produce more excellent and newer bodies, and can continue and develop spirit and character of oneself.

Creatures can keep self-identity even if constituent elements of creatures are different from those of preceding creatures, because, in the first place, ceatures always exchange their constituent elements by respiration and metabolism. Moreover, the consciousness of oneself can maintain continuity in the case that the infant self is lasting to the adult self, in the case that a particular self can put on weight and can lose weight as time goes by, and in the case that the cells, tissues, and organs of oneself were injured or lost in diseases, accidents, etc., and the self gets one's health back again. Namely, the self-continuity is preserved even if succeeding creatures have different shapes, sizes, functions, constituent elements, etc.

The phenomena that gene types of constituent elements are the same are recognized even on the level of cells, organelles, etc. The consciousness of oneself formed out of memories emerges on the level of individuals. The consciousness of oneself is very difficult to recognize on the level of cells and tissues, which is hard to present on the level of organs. The fact that the consciousness of oneself formed by memories emerges very clearly on the level of individuals appear even in identical twins. This invention can maintain formal identity and substantial development of the individual under the consciousness of the same self, by operating lots of identical organs synchronously, and by transferring various functions continuously.

The consciousness of oneself can expand and contract in the passage of time and on the spacial level. Consequently, both the infant self and the adult self can be connected as the same self. And so the spacial sensation of oneself can be extended freely on the occasion of handling transportation such as automobiles.

My invention can develop the consciousness of oneself in the individual of creatures with preserving the identity, by transferring minds and personalities of creatures to new clone bodies. Furthermore, uniting continuously and spaciously regenerated organs, such as new healthy cells, tissues, organs, which have the same gene types in new clone bodies, can rejuvenate oneself and can improve faculties of oneself. In addition to these repetitive processes, by using jointly artificial materials, new energy systems, etc., I can create diversely evolving lives.

1. This invention transfers minds and personalities of individuals and clone bodies of creatures to new clone bodies. And thus I can make creatures continue development permanently, with ensuring creatures existence bases of themselves.

2. In this way, with maintaining formal identity and substantial development of the individual eternally, I can rejuvenate and evolve structures and functions of individuals

3. By shifting some parts of an individual, especially parts with central nervous systems, to new clone bodies, an individual can get more excellent and newer bodies.

4. Putting both old and new brains into operation simultaneously and transferring functions gradually from old brains to new brains make it possible to realize self-continuity as time passes, without anxiety.

5. Securing existence bases of oneself in succession, and combining freely a great many of specific parts of central nervous systems and other systems by commissural fibers, association fibers, nerve fibers, etc., can increase memory capacity, with being free from small volume of the skull.

6. Moreover, by establishing new active pathways, and by producing more excellent and newer brains, I can enlarge and renew faculties of oneself. Remaking gray matters into chips such as semiconductors, various integrated circuits, etc., makes nervous systems light and convenient.

7. By securing plenty of nervous systems, such as basal ganglia, various nuclei, cerebella, brainstems, ganglia, etc., and by making the most of lots of other systems in addition to nervous systems at the same time, I can create the existence of multi-arms and multi-legs, which enlarges spacial self-continuity.

8. Fresh brains can be brought up, while coming into subsidiary operation, with carrying these immature brains on one's back. Free combinations of brains brought up in various environments broaden the world of one's own, which produce latest brains that have newly composed engrams. Incorporating these latest brains into central nervous systems once more leads to infinite development.

9. Creatures can maintain self-identity even if constituent elements of succeeding creatures are different from those of preceding creatures, because ceatures always exchange their constituent elements by respiration and metabolism.

10. Furthermore, the consciousness of oneself can maintain continuity in the case that the infant self is lasting to the adult self, in the case that a particular self can put on weight and can lose weight as time goes by, and in the case that the organs of oneself were injured in diseases, accidents, etc., and the self gets one's health back again. Namely, the self-continuity, i.e. the consciousness of oneself, is preserved even if succeeding creatures have different shapes, sizes, functions, constituent elements, etc.

11. The phenomena that gene types of constituent elements are the same are recognized even on the level of cells, organelles, etc. The consciousness of oneself formed out of memories emerges on the level of individuals. The consciousness of oneself is very difficult to recognize on the level of cells and tissues, which is hard to present on the level of organs. The fact that the consciousness of oneself formed by memories emerges very clearly on the level of individuals appear even in identical twins.

12. Therefore, with keeping the consciousness of the same self, connecting various organs and obtaining new organs, of which gene types are the same, can extend sphere of activity in daily life. Consequently, beyond a low capacity in the past, I can develop the faculties of all creatures including human beings and create diversely evolving lives.
